(19) United States
(12) Patent Application Publication
(10) Pub. No.: US 2005/0070748 A1
(43) Pub. Date: Mar. 31, 2005

(54) Process for production of styrene

(76) Inventors: Brian Ellis, Lower Sunbury (GB); Stephen Kenneth Pollitt, Cuffley (GB); Warren John Smith, Feltham (GB)

Correspondence Address: Finnegan Henderson Farabow;Garrett & Dunner, 1300 I Street NW, Washington, DC, 20005-3315, US

(21) Appl. No.: 10/498,054

(22) PCT Filed: Dec. 4, 2002
(86) PCT No.: PCT/GB02/05482

(30) Foreign Application Priority Data
Dec. 11, 2001 (GB) 0129611.0

(51) Int. Cl. C07C 2/00

(52) U.S. Cl. 585/323

(57) ABSTRACT

A process for the production of styrene is disclosed, comprising the steps of: a) feeding to an alkylation unit a stream of benzene and a stream of ethylene; b) mixing the outlet stream from the alkylation unit with a stream of ethane and a stream of oxygen; c) feeding the mixture obtained in b) to an oxodehydrogenation unit containing a catalyst capable of contemporaneously oxidatively dehydrogenating ethane and ethylbenzene to give ethylene and styrene respectively; d) feeding the product leaving the oxodehydrogenation unit to a separation unit to produce a stream containing styrene and a stream containing ethylene; e) recycling the stream containing ethylene to the alkylation unit.

The present invention relates to a process for the production of styrene, starting from benzene and ethane. More specifically, the present invention relates to a process for the production of styrene by the simultaneous oxodehydrogenation of ethylbenzene and ethane to give styrene and ethylene respectively.

As is well known, styrene is a product which is used in the production of thermoplastic polymers, such as polystyrenes (PS), acrylonitrile-butadiene-styrene copolymers (ABS), styrene-acrylonitrile resins (SAN), styrene-butadiene elastomeric copolymers (SBR) and in formulations for unsaturated polyester resins.

Styrene is generally prepared by the adiabatic or isothermic catalytic dehydrogenation of ethylbenzene in the presence of catalysts selected from metal oxides or their mixtures. In WO 9708034, for example, the catalyst consists of a mixture comprising Fe₂O₃, K₂O MnO₃, MgO, at least one oxide of Cu, Zn, Sc, Ti, W, Mn, Ni, Pd, Al, P, Bi, B, Sn, Pb and Si and at least two rare-earth metals. Further information on the dehydrogenation of ethylbenzene is available in Stanford Research Institute (SRI International) Report 338, 1977. Ethylbenzene is, in turn, prepared by the alkylation of benzene, available as a refinery product, with ethylene typically coming from the cracking or dehydrogenation of ethane. Details on the alkylation of benzene with ethylene are available in SRI.

In EP 905112A, a process for the simultaneous dehydrogenation of ethylbenzene and ethane to produce ethylene and styrene is disclosed. The process comprises:
a) feeding to an alkylation unit a stream of benzene and a stream of recycled product containing ethylene;
b) mixing the stream at the outlet of the alkylation unit, containing ethylbenzene, with a stream consisting of ethane;
c) feeding the mixture thus obtained to a dehydrogenation unit containing a catalyst capable of contemporaneously dehydrogenating ethane and ethylbenzene to give ethylene and styrene respectively;
d) feeding the product leaving the dehydrogenation unit to a separation section to produce a stream essentially consisting of styrene and a stream containing ethylene;
e) recycling the stream containing ethylene to the alkylation unit.

The dehydrogenation of ethylbenzene is a highly endothermic reaction, requiring severe conditions. As a consequence, the above process is energy intensive and has high utility costs. We have now discovered that these problems can be reduced by replacing the dehydrogenation reaction with an oxidative dehydrogenation (oxodehydrogenation) reaction.

Accordingly in a first aspect of the present invention provides a process for the production of styrene, comprising the steps of:
a) feeding to an alkylation unit a stream of benzene and a stream of ethylene;
b) mixing the outlet stream from the alkylation unit with a stream of ethane and a stream of oxygen;
c) feeding the mixture obtained in b) to an oxodehydrogenation unit containing a catalyst capable of contemporaneously oxidatively dehydrogenating ethane and ethylbenzene to give ethylene and styrene respectively;
d) feeding the product leaving the oxodehydrogenation unit to a separation unit to produce a stream containing styrene and a stream containing ethylene;
e) recycling the stream containing ethylene to the alkylation unit.

We have found that the above process results in a longer lasting catalyst, as a consequence of the less severe conditions than in the prior art process, and also the presence of oxygen, which reduces coking.

Typically the ethylene-containing stream exiting the separation unit also contains a significant proportion of unreacted ethane. In one embodiment, the ethylene and ethane are separated prior to the ethylene being recycled to the alkylation unit.

According to the simplest concept of the invention, a first stream of benzene is fed to the alkylation unit, together with a second stream of recycled product, essentially consisting of ethylene and non-converted ethane, with over 50 weight % usually being non-converted ethane. Typically, this second stream comprises 2-20% by weight of ethylene and 80-98% by weight of ethane, together with about 0.1-1% by weight (calculated out of the total of ethylene+ethane) of other light products, formed in both the alkylation and dehydrogenation phase.

The two streams are fed to the alkylation unit to give a benzene/ethylene ratio of typically between 3 and 10, more typically 6-8. The alkylation reaction is carried out in a conventional reactive distillation process, such as described for example in EP 432814A. The alkylation unit is typically operated at a temperature of between 250 and 450° C., preferably 350-400° C.; and at 1-30 bar, preferably 15-20 bar pressure. In addition to the reactive distillation column, the alkylation unit may additionally comprise a fixed bed liquid phase alkylation reactor for treating the products from the reactive distillation column. A transalkylation unit to convert diethylbenzene and triethylbenzene to ethylbenzene is typically also present.

The ethylbenzene product from the alkylation unit is mixed with ethane, which can be fresh ethane or can comprise a mixture of fresh and recycled ethane. Oxygen is also introduced as the stream is fed into the oxodehydrogenation unit, either as a single stream or at several injection points along the catalyst bed. Recycled ethylbenzene may also be added at this point. To obtain a good balance between the alkylation and dehydrogenation reactions it is preferable for the total ethane, both recycled and fresh, to be present in such an amount is to give molar ratios of ethylbenzene to ethane of between 0.05 and 10, preferably 0.1 and 1. Oxygen levels are generally 2-20 mol % and more preferably 6-12 mol % in the inlet stream. The oxygen may be introduced in the form of a molecular oxygen-containing gas, which may be air or a gas richer or poorer in molecular oxygen than air, for example pure oxygen. A suitable gas may be, for example, oxygen diluted with a suitable diluent, for example nitrogen or helium.

The dehydrogenation reaction is preferably carried out in gaseous phase operating in fixed-bed, moving-bed or fluid-bed catalytic reactors, although fluid-bed reactors are preferred for their technological advantages which are well known to experts in the field.

Any catalyst capable of contemporaneously oxidatively dehydrogenating a paraffin such as ethane and an alkylaromatic hydrocarbon such as ethylbenzene can be used in the oxodehydrogenation reaction. Particularly preferred are those catalysts disclosed in our own EP 1043064A. They comprise in combination with oxygen the elements molybdenum, vanadium, niobium and gold according to the empirical formula:
MoₐW_{b}Au_{c}V_{d}NbₑY_{f} (I)
wherein Y is one or more elements selected from the group consisting of: Cr, Mn, Ta, Ti, B, Al, Ga, In, Pt, Pd; Zn, Cd, Bi, Ce, Co, Rh, Ir, Cu, Ag, Fe, Ru, Os, K, Rb, Cs, Mg, Ca, Sr, Ba, Zr, Hf, Ni, P, Pb, Sb, Si, Sn, Tl, U, Re, Te and La;
a, b, c, d, e and f represent the gram atom ratios of the elements such that:
0<a≦1; 0≦b<1 and a+b=1;
10⁻⁵<c≦0.02;
0<d≦2;
0<e≦1; and
0≦f≦2.

Preferably Y does not include Pd.

Catalysts embraced within the formula (I) include:
MoₐW_{b}Au_{c}V_{d}NbₑY_{f}
Mo_{a.}Au_{c}V_{d}NbₑY_{f}
MoₐW_{b.}Au_{c}V_{d}Nbₑ
Mo_{a.}Au_{c}V_{d}Nbₑ

Examples of suitable catalysts having the formula (I) include:
Mo_{1.00}V_{0.25}Nb_{0.12.}Au_{0.01}O_{y}; Mo_{1.00}V_{0.213}Nb_{0.138}Au_{0.007}O_{y}; Mo_{1.00}V_{0.232}Nb_{0.139}Au_{0.007}O_{y}; and Mo_{1.000}V_{0.426}Nb_{0.115}Au_{0.0008}O_{y} wherein y is a number which satisfies the valencies of the elements in the composition for oxygen.

Preferably a>0.01. Preferably, d>0.1. Preferably, e>0.01. Preferably, e≦0.5. Preferably, f≧0.01. Preferably, f≦0.5.

Preferably, Y is selected from the group consisting of Bi, Ca, Ce, Cu, K, P, Sb, La and Te.

In the fluid-bed dehydrogenation reactor, it is preferable to operate:
at a temperature ranging from 300 to 550° C., more preferably 350-400° C.,
at a pressure of from 1 to 30 bar and more preferably in the range 10-20 bar; at a gas hourly space velocity of between 2000-6000/h preferably between 3000-4000/h with a residence time of the catalyst in the fluid-bed zone varying from 1 to 60 seconds, preferably from 5 to 10 seconds.

At the end of the oxodehydrogenation reaction, a dehydrogenated stream is recovered, typically comprising: 2-35%, more typically 5-15% by weight of styrene; 1-20%, more typically 5-15% of ethylene; 25-75%, more typically 40-50% of non-reacted ethane and 2-40%, more typically 10-30% of non-reacted ethylbenzene; 0.1-2% of other products such as methane, hydrogen, toluene, benzene and possibly acetic acid formied during both the alkylation and dehydrogenation reaction. This stream is passed to a degasifier, and then to a decanter where water and water-soluble products are removed. The hydrocarbon liquid portion is then separated into benzene, recycled to the alkylation unit, ethylbenzene, which is recycled to the oxodehydrogenation unit, and styrene which is collected. In a preferred embodiment, the gaseous portion comprising ethylene and possibly unreacted ethane is passed through a COₓ removal unit; the ethylene/ethane stream is then recycled to the alkylation unit. If acetic acid is present in the dehydrogenated stream, this may optionally be recovered as a separate product. In any case, where acetic acid is present it is necessary to ensure that the metallurgy of the system is suitable, with higher grade alloy or stainless steel being used.

Two specific embodiments of the invention are now described, with reference to the accompanying drawings, in which:

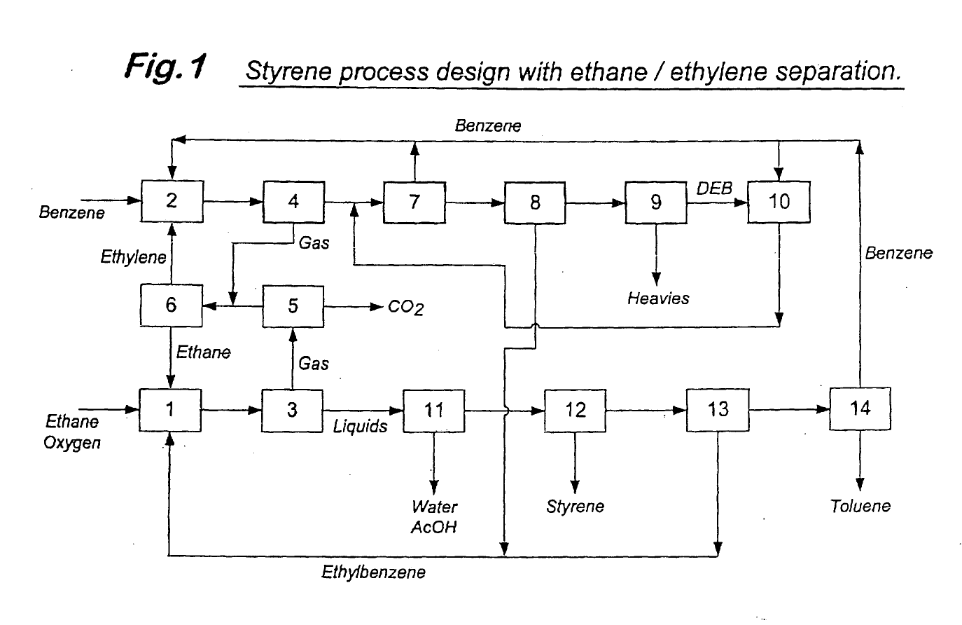

FIG. 1 is a flow chart of the first example, and

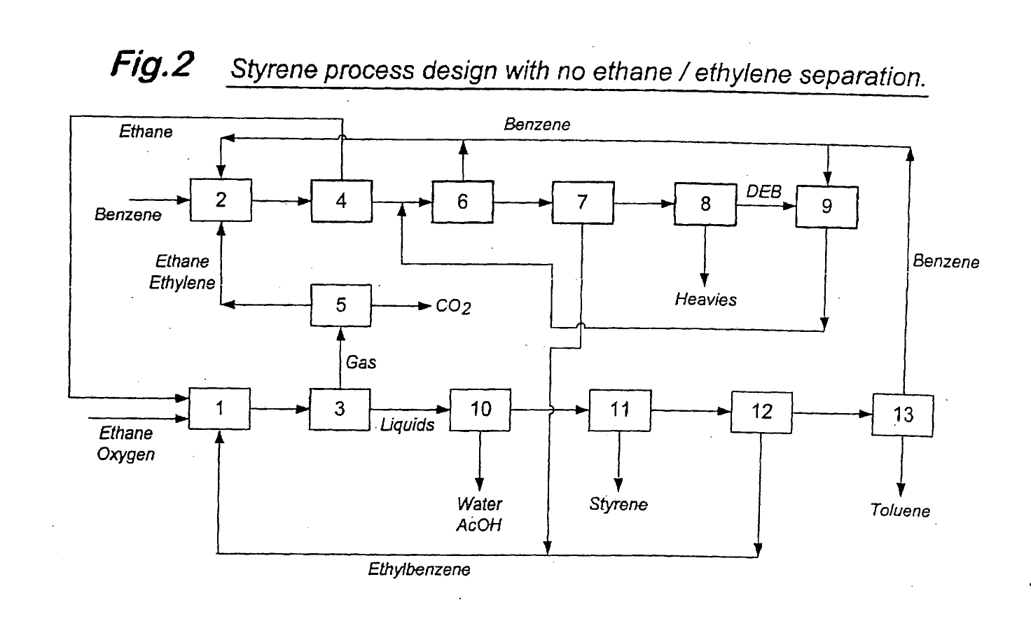

FIG. 2 is a flow chart of the second example.

In a first example of the process (FIG. 1), an oxydehydrogenator (1) is operated at 300-550° C. and 1-30 bar pressure to simultaneously convert ethane to ethylene and ethylbenzene to styrene. A second reactor, the alkylator (2) is operated at 250-450° C. and 1-30 bar pressure to alkylate benzene with ethylene. In one embodiment of the process, the products from the oxydehydrogenator (1) are fed to a degasifier unit (3), with the recovered gaseous products being fed to a common COₓ removal unit (5) before passing to an ethane/ethylene separation unit (6). The latter can be of the Selective Olefin Recovery type (SOR), cryogenic type, or any other type. Following ethane/ethylene separation, the recovered ethane is recycled to the oxydehydrogenator (1), while the ethylene is recycled to the alkylator (2). The products from the alkylator (2) are fed to a separate degasifier (4), with the recovered gases being fed to the ethanelethylene separation unit (6). The liquids from the alkylator degasifier (4) are sent to a benzene recovery column (7), where the recovered benzene is optionally dried in a drying column before being recycled to the alkylator (2). The liquids separated from the benzene in (7) are passed to a column (8) where ethylbenzene is recovered and recycled to the oxydehydrogenator (1). The liquids separated from the ethylbenzene in (8) are fed to a column (9) where DEB is recovered from polyalkylate heavy residue.

The recovered DEB from (9) is passed to a transalkylator unit (10) where it is reacted with benzene from the recycle stream to produce ethylbenzene which is recycled to the benzene recovery column (6). The liquids separated from the gas in (3) are passed to a decanter (11), where water and water-soluble products such as acetic acid are recovered, the residual organic liquids separated in (11) being passed to a column (12) where styrene is recovered. The liquids separated from styrene in (12) are sent to a column (13) where ethylbenzene is recovered and recycled to the oxydehydrogenator (1). The liquids separated from ethylbenzene in (13) are then passed to a column (14) where trace levels of benzene are separated from toluene overhead and recycled to the alkylator (2).

In this example of the process, it is preferable to operate to 100% oxygen depletion in the oxydehydrogenator (1), in order to simplify the degasification and ethane/ethylene separation processes.

In a second example of the process (FIG. 2) an oxydehydrogenator (1) is operated at 300-550° C. and 1-30 bar pressure to simultaneously convert ethane to ethylene and ethylbenzene to styrene. A second reactor, the alkylator (2) is operated at 250-450° C. and 1-30 bar pressure to alkylate benzene with ethylene. It is a key feature of this example of the proposed process that no ethane/ethylene separation stage is required due to the use of the following configuration. The products from the oxydehydrogenator (1) are passed to a degasifier (3) before feeding to a COₓ removal unit (5), after which the gaseous effluent consisting of ethylene diluted in ethane is fed directly to the alkylator (2)—the alkylator being able to process low purity ethylene feedstocks as exemplified by catalytic distillation units. Conversely, the exit stream from the alkylator (2) is fed to a separate degasifier (4) and the gaseous stream consisting mainly of ethane is then passed directly to the oxydehydrogenator (1). The liquids from the alkylator degasifier (4) are sent first to a benzene recovery column (6), where the recovered benzene is optionally dried in a drying column before being recycled to the alkylator (2). The liquids separated from the benzene in (6) are passed to a column (7) where ethylbenzene is recovered and recycled to the oxydehydrogenator (1). The liquids separated from the ethylbenzene in (7) are fed to a column (8) where DEB is recovered overhead from the polyalkylate heavy residue. The recovered DEB from (8) is passed to a transalkylator unit (9) where it is reacted with benzene from the recycle stream to produce ethylbenzene which is recycled to the benzene recovery column (7). The liquids separated from the gas in (3) are passed to a decanter (10), where water and water-soluble products such as acetic acid are recovered, the residual organic liquids being passed to a column (11) where styrene is recovered. The liquids separated from styrene in (11) are sent to a column (12) where ethylbenzene is recovered and recycled to the oxydehydrogenator (1). The liquids separated from ethylbenzene in (12) are then passed to a column (13) where trace levels of benzene are separated from toluene overhead and recycled to the alkylator (2).

In this example of the process, it is preferable to operate to 100% oxygen depletion in the oxydehydrogenator (1) and to 100% ethylene depletion in the alkylator (2), thus simplying the degasification and avoiding the need for an ethane/ethylene separation unit.

1. Process for the production of styrene, comprising the steps of:
a) feeding to an alkylation unit a stream of benzene and a stream of ethylene;
b) mixing the outlet stream from the alkylation unit with a stream of ethane and a stream of oxygen;
c) feeding the mixture obtained in b) to an oxodehydrogenation unit containing a catalyst capable of contemporaneously oxidatively dehydrogenating ethane and ethylbenzene to give ethylene and styrene respectively;
d) feeding the product leaving the oxodehydrogenation unit to a separation unit to produce a stream containing styrene and a stream containing ethylene;
e) recycling the stream containing ethylene to the alkylation unit.

2. Process according to claim 1, wherein in step (a) a first stream of benzene is fed to the alkylation unit, together with a second stream of recycled product, comprising 2-20% by weight of ethylene, 80-98% by weight of non-converted ethane, and 0.1-1% by weight (based on the total of ethylene+ethane) of other light products.

3. Process according to claim 1 or 2, wherein the benzene/ethylene ratio in the alkylation unit is between 3 and 10, preferably between 6 and 8.

4. Process according to any preceding claim, wherein in the oxodehydrogenation unit the molar ratio of ethylbenzene to ethane is between 0.05 and 10, preferably between 0.1 and 1.

5. Process according to any preceding claim, wherein the level of oxygen in the inlet stream to the oxodehydrogenation unit is 2-20 mol %, preferably 6-12 mol %.

6. Process according to any preceding claim, wherein the catalyst for oxidatively dehydrogenating ethane and ethylbenzene has the empirical formula:
MoₐW_{b}Au_{c}V_{d}Nb_{c}Y_{f} (I)
wherein Y is one or more elements selected from the group consisting of: Cr, Mn, Ta, Ti, B, Al, Ga, In, Pt, Pd, Zn, Cd, Bi, Ce, Co, Rh, Ir, Cu, Ag, Fe, Ru, Os, K, Rb, Cs, Mg, Ca, Sr, Ba, Zr, Hf, Ni, P, Pb, Sb, Si, Sn, Tl, U, Re, Te and La;
a, b, c, d, e and f represent the gram atom ratios of the elements such that
0<a≦1; 0≦b<1 and a+b=1;
10⁻⁵<c≦0.02;
0<d≦2;
0<e≦1;
0<f≦2; and
preferably Y does not include Pd.

7. Process according to any preceding claim, wherein the stream leaving the oxodehydrogenation unit comprises 2-35%, preferably 5-15% by weight of styrene; 1-20%, preferably 5-15% of ethylene; 25-75%, preferably 40-50% of non-reacted ethane and 2-40%, preferably 10-30% of non-reacted ethylbenzene; and 0.1-2% of other products.

8. Process according to claim 7, wherein the hydrocarbon liquid portion of the stream leaving the oxodehydrogenation unit is separated into benzene, ethylbenzene and styrene, and the gaseous portion is passed through a COₓ removal unit and the resulting ethylene/ethane stream recycled to the alkylation unit.
